(19) United States
(12) Patent Application Publication
(10) Pub. No.: US 2004/0116895 A1
(43) Pub. Date: Jun. 17, 2004

(54) Device for inserting a needle-shaped body into living tissue

(76) Inventors: Ulrich Werth, Magdeburg (DE); Karl-Otto Launicke, Markneukirchen (DE)

Correspondence Address: Pendorf & Cutliff, 5111 Memorial Highway, Tampa, FL, 33634-7356, US

(21) Appl. No.: 10/470,513

(22) PCT Filed: Jan. 28, 2002
(86) PCT No.: PCT/DE02/00351

(30) Foreign Application Priority Data
Jan. 29, 2001 (DE) 101 04 929.3
Feb. 28, 2001 (DE) 101 10 513.4

(51) Int. Cl. A61M 31/00; A61K 9/22

(52) U.S. Cl. 604/506; 604/890.1; 604/59

(57) ABSTRACT

The invention relates to a needle-type body embodied in the form of a rigid implant (1) having a tip (2) and provided with a guide surface (4) disposed thereon and/or a shank (3) which is joined to the tip and provided with a guide surface (5) and/or the implant has a cylindrical or spherical body. The aim of the invention is to insert said body into living tissue so that the implant is fully surrounded by tissue, whereby the point of insertion can thus heal. In order to insert the implant (1) into the tissue, a cartridge and tube combination is used, consisting of a tube (6) which has a continuous borehole (7) into which the implant can be inserted and through which the implant (1) is guided, in addition to a plunger (8) which is displaceably arranged in the bore hole (7) and whose length is greater than the depth of the tube (6). The tube (6) is embodied in the form of a truncated cone or a truncated pyramid on the end where the implant (1) can be expelled. This makes it easier to place the tube (6) onto tissue with greater precision.

The invention relates to a needle-type body, which in particular can be introduced into human skin tissue.

From the Chinese acupuncture it is known to pierce metallic needles into skin points in the skin as determined according to a template. The acupuncture needles have a length of up to 150 mm.

The disadvantage of such needles consists therein that the person becomes incapable of acting due to the acupuncture needles pierced into the skin.

In addition to the classic Chinese acupuncture an ear acupuncture, probably having a western or Arabic origin, during the last years has become of interest in Western Europe. Also a stimulation by means of needles takes place here but with the abovementioned disadvantages. Short needles were developed to assist, at the shank of which needles a thickening is present, which limits insertion into the skin. Such a needle and a needle insertion device are described in the DE 26 57 053 A1.

This device is characterized by a needle with a conical tip and a flat head, by a hollow body, which can be pressed against the tissue or the skin of a patient, and in which the needle is displaceably arranged, by a member arranged displaceably in the hollow body for ejection of the needle from the hollow body into the tissue or the skin of a patient, and by means allocated to the hollow body as well as the displaceable member for causing the displaceable member to leave it to drive the needle out of the hollow body.

The disadvantage of this type of needle is that, due to unintended touching of the outer lying thickening, pain is caused and tearing out is not avoided.

Furthermore in connection with earrings and piercing, it is known to pierce through the skin, to introduce a sticker into the opening and to prevent this from slipping out by way of a counter holder or to lock this after guiding through the opening. Here there also is the danger of tearing in or tearing out of the skin on unintended touching.

Object of the invention is to introduce a needle-type body in such a manner into living tissue that an injury of the tissue by way of subsequent touching of the needle-type body is excluded.

This object is achieved by means of the characterizing features of the method claim 1 and the device claim 21. Uses are contained in the claims 32 to 34. Advantageous embodiments are the subject of the subsidiary claims.

According to the invention a needle-type body, which is designed as a rigid is implant with a tip and a guide surface disposed thereto and/or a shank with guide surface joining onto the tip, whereby the guide surfaces are disposed and formed such that they slidingly guide the implant in a tube, prior to insertion into the tissue, is thus inserted into living tissue that the tissue surrounds the implant fully. The piercing position then can heal.

Similar to a conventional acupuncture needle the implant can be pressed at an acupuncture point through the skin surface and remains there so that, remaining with the example of the acupuncture, a self-healing potential is activated permanently by permanent irritation.

The tip of the implant is, in accordance with the invention, formed arrow-shaped or is a cone or a three or multiple-sided pyramid or has a tear shaped, heart shaped or screw shaped form. Thereby the shell of an arrow, conical or pyramid shaped peak advantageously has a slight curvature.

The shank of the implant preferably is cylindrically shaped and smooth at the tip, whereby its circumferential dimension does not exceed that of the tip relative to the common central axis. The jacket of the cylindrically shaped shank can have grooves for stabilising seating in the tissue.

The implant has a length L of less than 5 mm in a preferred embodiment and the largest thickness dimension of the tip and/or the shank should also not exceed 5 mm.

According to an embodiment the guide surface at the tip extends about this ring-shaped at the largest circumference and is designed to be cylindrically shaped. Another variation envisages several guide surfaces at the tip at the circumference in insertion direction so that the tip can be guided slidingly in a tube prior to insertion into the tissue. In a further variation in place of the guide surface of the tip a bore is provided on the side of the peak facing away from the insertion position into which a pin is form tightly and releasably insertable for insertion into the tissue.

In a further embodiment it is provided that the implant is formed as plain cylindrically shaped body or cylinder with straight flat planes extending at the circumference in cylindrical longitudinal direction or as sphere or sphere-like body, also with flat planes at the surface. This embodiment also can be inserted without pain into the tissue provided the size is selected correspondingly small. The diameter of the cylindrical body should be less than 1 mm and the diameter of the sphere or the sphere-like body also less than 1 mm. The cylindrical jacket can have recesses or grooves to ensure a stable location of the cylindrically-shaped body, which is enclosed fully in the tissue.

Metallic materials, in particular stainless steel, also gold plated or silver plated, have been found suitable as material for the implant. However, the invention is not restricted thereto.

The method in accordance with the invention, in which a needle-type body is inserted into living tissue, provides that the body, formed as rigid implant, is inserted completely into the living tissue starting with the tip so that after removal of the insertion tool it is completely surrounded by the tissue. The insertion position then can heal together.

In the described implant size it is of advantage to use a device for insertion of the implant into the living tissue. This consists in an embodiment in accordance with the invention of a tube with a continuous bore into which the implant is insertable and through which the implant is guided as well as a plunger arranged displaceably in the bore for pressing out the implant. The plunger has a length which is larger than the depth of the tube so that, on placing the tube onto the tissue, it not only presses the implant out of the tube and into the tissue but itself also can penetrate into the tissue. After the implant has been positioned, the plunger then is withdrawn and the opening in the living tissue then can draw together and heal.

It is advantageous if the tube is shaped as truncated cone or truncated pyramid at the end, where the implant is pushed out by the plunger in order to ensure an exact placing of the tube onto the tissue.

Obviously it would be possible to move the plunger directly by hand when placing the implant. However, due to the small dimensions it is advantageous if the tube is movable in an one-sided open cartridge, the opening of the cartridge being on the side where the implant is pushed out of the tube, and the plunger of the tube is arranged inside at the floor of the cartridge, whereby its length is dimensioned such that it projects sideways beyond the cartridge and the fully pushed in tube. The movement of the plunger in the tube is achieved by means of the relative movement between the cartridge and the tube and the plunger length ensures pushing the implant completely into the tissue.

In a further embodiment of the invention it is provided that the outer floor of the cartridge has a recess, which is dimensioned such that it can serve as receptor for a section of the tube projecting out of another cartridge during placement insertion of the implant in the tube. Therewith the condition exists to provide several cartridges with tubes in series in a magazine and to push these out of the cartridge tube individually one after the other. This offers a considerable advantage regarding reduction of the application times, if several implants have to be applied one after another.

Such a cartridge tube has a stiff jacket and in a particular embodiment, is elastically designed in the outlet region of the cartridges or a region in front of it. Thus the cartridge situated in this region can be fixed by finger pressure on the setting end of the implant for placing the implant from outside so that the tube of the cartridge, which is situated at the cartridge tube outlet, after placing onto the tissue and pressing towards the tissue, is pushed into the cartridge and thereby the plunger of this cartridge pushes the implant out of the tube and into the tissue.

Alternatively or simultaneously the possibility exists to dispose at least one support in the cartridge tube, which allows a movement of one or more cartridges towards the outlet region but prevents movement away from the outlet region into the cartridge tube.

Similarly in a further embodiment the invention provides that the cartridge tube has a feeding arrangement for at least one cartridge, which is operable from outside. Also in this manner the cartridge with tube can be brought into the application position and by way of the relative movement between the front cartridge and the tube located in it and therewith the pushing out of the implant can be achieved.

It furthermore has been shown that such a cartridge-tube-combination can be arranged releasably advantageously at a pincer leg, whereby the tube outlet for the implant is directed towards of the other pincer leg. This should be formed in the front region to be flat, plate-shaped, or convex or concave curved or be adapted to the tissue form and on pressing in the implant to support the tissue on the opposite side. Thus the implant can be introduced in advantageous manner into flat tissue parts, such as ear, tongue, nose or skin folds or also thin limbs

If several implants are to be applied one after the other, then it is provided in accordance with the invention that, in place of a cartridge-tube-combination, a cartridge tube, which contains several cartridge-tube-combinations, are disposed at a pincer leg. The other pincer leg here is also formed flat, plate shaped, or also convex or concave curved or adapted to the tissue form, so that it can support the tissue on the opposite side on pressing in the implant.

Examples of embodiments of the invention are shown in the drawings. It is shown in:

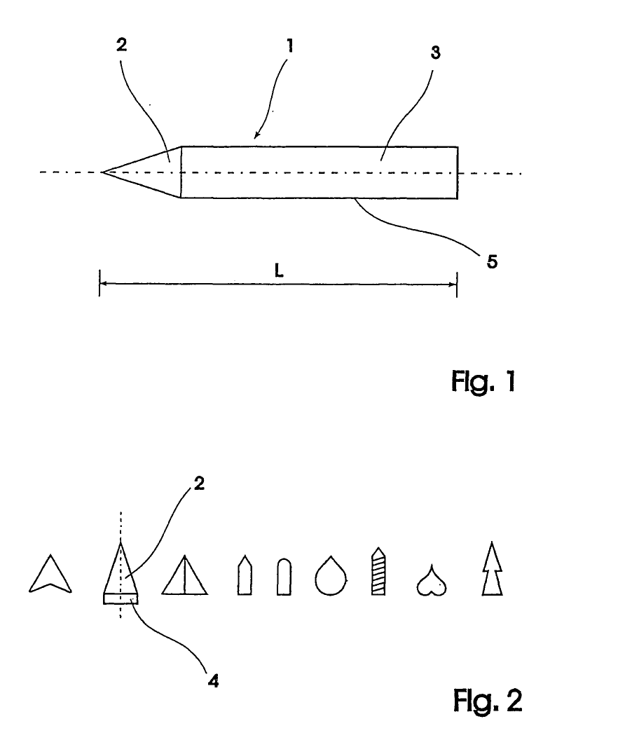

FIG. 1 an implant with shank,

FIG. 2 tips of implants,

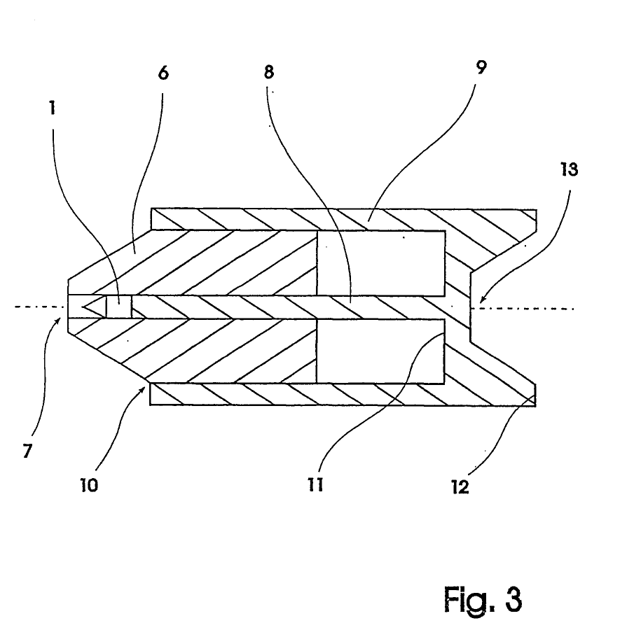

FIG. 3 a cartridge-tube-combination,

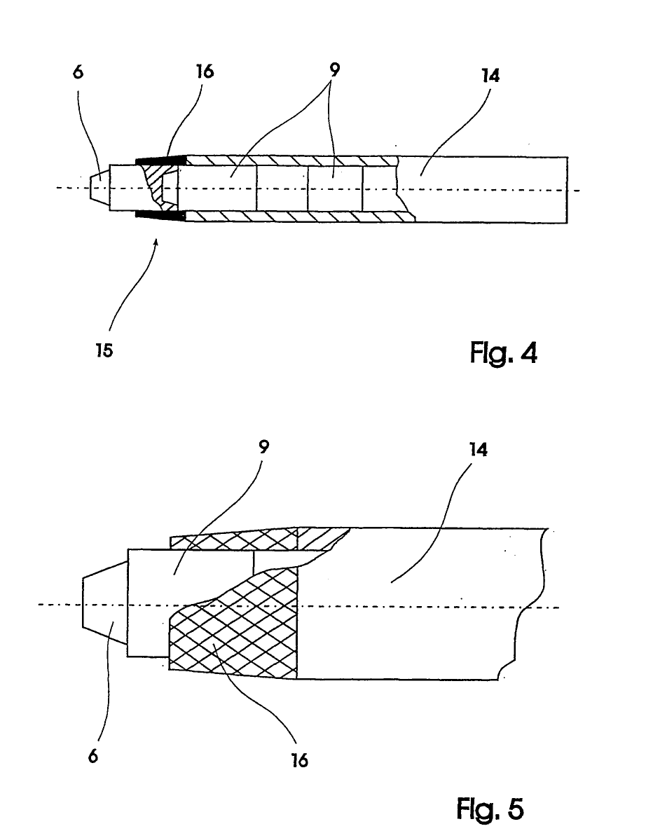

FIG. 4 several cartridges and tubes in a cartridge tube,

FIG. 5 the front part of a cartridge tube,

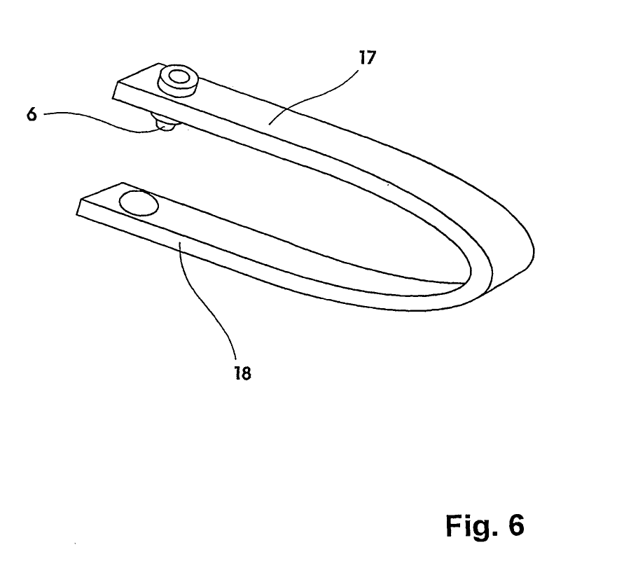

FIG. 6 pincer with cartridge-tubes-combination.

The rigid implant in accordance with the invention shown in FIG. 1 consists of a tip 2 and a shank 3. The size is dimensioned such that, after insertion into the tissue, it is fully enclosed by the tissue and can heal. If the implant is introduced such a manner into human skin that the pin 2 meets at an acupuncture point, then a self-healing potential can be activated permanently. A dimension smaller than 5 mm from the pin 2 up to the end of the shank 3 has been proven to be worthwhile as length and the largest thickness dimension of the pin 2 and/or the shank 3 also should not exceed 5 mm. Metallic materials are in particular applicable as construction materials for the implants 1.

In FIG. 2 embodiment forms of tips 2 of the implants 1 are illustrated. The tips 2 preferably are designed arrow-shaped or represent a cone or a three or multi-sided pyramid or have a tear shaped, heart shaped or screw shaped form. The shell of an arrow, cone or pyramid shaped peak thereby can have a slight curvature.

FIG. 3 shows a cartridge-tubes-combination for insertion of the implant 1 into the tissue. It consists of a tube 6 with continuous bore 7 into which the implant 1 is insertable and through which the implant 1 is guided, as well as a plunger 8 arranged displaceably in the bore 7, the length of which is larger than the depth of the tube 6. The tube 6 is truncated conically shaped or truncated pyramid shaped at the end where the implant 1 is pushed out by the plunger 8. This supports an exact placement of the tube 6 onto the tissue.

The tube 6 is located displaceably in the cartridge 9, which is open on one side. The opening 10 of the cartridge 9 is located on that side where the implant 1 is pushed out of the tube 6. The plunger 8 of the tube 6 is arranged on the inside at the floor 11 of the cartridge 9, whereby its length is dimensioned such that it projects sideways beyond of the cartridge 9 and the completely inserted tube 6. The movement of the plunger 8 in the tube 6 thus is achieved by the relative movement of the cartridge 9 and the tube 6. The cartridge-tubes-combination with inserted implant 9 therewith forms a closed industrially produceable and sterile storeable unit.

Furthermore in this embodiment it is shown that the outer floor 12 of the cartridge 9 has a recess 13, which is dimensioned such that it can serve as reception for a section of the tube 6 projecting out of another cartridge 9.1 when an implant 1 is inserted into the tube 6. Thus optimally several cartridges 9.n with tubes 6 can be magazinized one after another in a cartridge tube 14, in order to push them one after another individually out of the cartridge tube.

FIG. 4 shows several cartridges 9-9-n with tubes 6 magazinized in a cartridge tube 14. The cartridges 9 thereby are arranged to be pushed individually out of the cartridge tube 14. The cartridge tube 14 has a stiff jacket, which has an elastic partial region 16 in the outlet region 15 or in front of it. This is shown in detail in FIG. 5. The cartridge 9 present in this partial region 16 can be fixed thus for placing the implant 1 by finger pressure from outside on the setting end, whereby the tube 6 of the cartridge 9 located at the cartridge tube outlet, after placement onto the tissue and being pressed towards the tissue, pushes into the cartridge 9 and thereby moves the plunger 8, which, due to its length, pushes the implant 1 completely out of the tube 6 and into the tissue.

FIG. 6 shows the cartridge-tubes-combination arranged in a pincer. In the pincer with the pincer legs 17, 18 by finger pressure, the cartridge-tubes-combination with the tube 6 is releasably arranged at the pincer leg 17, whereby the tube outlet for the implant shows towards the other pincer leg 18. The front region of the pincer leg 18 is plate-shaped. Thus it also can support the tissue on pressing in the implant.

In particular during pressing-in of the implant into the ear it can be useful to provide the pincer leg 18 in the front region with a form substantially adapted to a negative form of an ear. In place of the pincer shown here also a pincer with crossing pincer legs can be used.

In addition to the mentioned use of such implants during acupuncture further application possibilities are possible, e.g. in non-visible identification of animals. The implant 1, preferably the shank 3, should be provided with a readable code before insertion. In practice this can be realized by way of passive transponder technology because these function without current supply and can be read by way of a reading device at any time without contact by radio waves. Perspectively such implants also can be used for location purposes.

The low frequency transponders today applied in electronic price labels and ski passes have about one meter range, modern high frequency systems can be read up to 30 meter distance.

### REFERENCE NUMERALS

1 Implant

2 Tip of implant

3 Shank of implant

4 Guide surface tip

5 Guide surface shank

6 Tube

7 Bore

8 Plunger

9 Cartridge

10 Opening

11 Inner floor of cartridge

12 Outer floor

13 Recess

14 Cartridge tube

15 Outlet region

16 Partial region of cartridge tube

17 Pincer leg

18 Pincer leg

What is claimed is:

1. Method by means of which a needle-type body is inserted into living tissue, characterized thereby that
commencing with the peak (2) the body formed as stiff implant (1) is initially completely inserted into the living tissue, so that it is completely enclosed by the tissue after removal of the insertion tool.

2. Method according to claim 1, characterized thereby that
the insertion position heals together.

3. Method according to claim 1 or 2, characterized thereby that
the introduction of the implant (1) takes place into flat tissue parts, such as ear, tongue, nose or skin folds as well as into thin limbs.

4. Method according to one of the claims 1 to 3, characterized thereby that
a stiff implant (1) with a tip (2) and a guide surface (4) disposed thereon and/or a shank (3) joining onto the tip (2) with guide surface (5), is whereby the guide surfaces (4, 5) are formed such that they allow the implant (I) to be slidingly guided in a tube before insertion into the tissue.

5. Method according to one of the claims 1 to 4, characterized thereby that
an implant (1) is used, of which the tip (2) is shaped arrow-shaped or represents a conical or three or multiple-sided pyramid or has a tear shaped, heart shaped or screw shaped form.

6. Method according to one of the claims 1 to 5, characterized thereby that
an implant (1) is used of which its shell of an arrow, conical or pyramid shaped tip (2) has a slight curvature.

7. Method according to one of the claims 1 to 5, characterized thereby that
an implant (1) is used of which its shank (3) is cylindrically shaped formed onto the tip (2) whereby its circumferential dimension does not exceed that of the tip (2) relative to the common centre axis.

8. Method according to one of the claims 1 to 7, characterized thereby that
an implant (1) is used of which the jacket of the cylindrical shank (3) has grooves.

9. Method according to one of the claims 1 to 8, characterized thereby that
an implant (1) is used of which the shank (3) is smoothly formed onto the tip (2).

10. Method according to at least one of the claims 1 to 9, characterized is thereby that
an implant (1), which has a readable code, preferably in the shank (3) is used.

11. Method according to one of the claims 1 to 10, characterized thereby that
an implant (1), of which its length L is smaller than 5 mm, is used.

12. Method according to one of the claims 1 to 11, characterized thereby that
an implant (1), of which the largest thickness dimension of the tip (2) and/or the shank (3) does not exceed 5 mm, is used.

13. Method according to one of the claims 1 to 12, characterized thereby that
an implant (1), of which the guiding surface (4) at the tip (2) extends ring-shaped at the largest circumference and is cylindrically shaped, is used.

14. Method according to one of claims 1 to 13, characterized thereby that
an implant (1), which has several guide surfaces at the tip (2) extending circumferentially in insertion direction, is used so that the tip (2) can be slidingly guided in a tube prior to insertion into the tissue.

15. Method according to one of the claims 1 to 4, characterized thereby that
an implant (1), which has a bore on the side of the tip (2) facing away from the insertion position and a pin form tightly and releasably inserted into the bore for insertion into the tissue, is used.

16. Method by means of which a needle-shaped body is inserted into living tissue, characterized thereby that
an implant (1), which is designed as thin cylindrically shaped body or cylinder with straight planes extending circumferentially in the cylindrical longitudinal direction or as sphere or sphere-like body; also with flat planes at the surfaces and in a small diameter, and is inserted fully into the living tissue, is used.

17. Method according to claim 16, characterized thereby that
an implant (1), in which the diameter of the cylindrical body is smaller than 1 mm, is used.

18. Method according to claim 16 or 17, characterized thereby that
an implant (1), in which the diameter of the sphere or sphere-like body is smaller than 1 mm, is used.

19. Method according to one of the claims 16 to 18, characterized thereby that
an implant (1), in which the cylinder jacket has recesses or grooves, is used.

20. Method according to one of the claims 1 to 19, characterized thereby that
an implant (1), which consists of a metallic material, in particular stainless steel, gold-plated or silver-plated, is used.

21. Device for performing the method according to claim 1 or 16 consisting of a tube (6) with a continuous-bore (7) into which the implant (I) is insertable and through which the implant (I) is guided as well as a plunger (8) displaceably arranged in the bore (7), of which the length is larger than the thickness of the tube (6).

22. Device according to claim 21, characterized thereby that
the tube (6) is shaped conically truncated or pyramid truncated at the end, where the implant (1) is to be pushed out by the plunger (8).

23. Device according to claim 21 or 22, characterized thereby that
the tube (6) is mounted displaceably in a one side open cartridge (9), the opening (10) of the cartridge (9) being on the side where the implant (1) can be pushed out of the tube (6), and the plunger (8) of the tube (6) is arranged inside at the floor (11) of the cartridge (9) of the cartridge (8), whereby its length is dimensioned such that it projects beyond the cartridge (9) and into the completely pushed-in tube (6).

24. Device according to claim 23, characterized thereby that
the outer floor (12) of the cartridge (9) has a recess (13), which is dimensioned such that it serves as reception for a section of the tube (6) projecting beyond another cartridge (9.1) on inserted implant (1) in the tube (6).

25. Device according to claim 24, characterized thereby that
several cartridges (9-9.n) tubes (6) are magazinized in a cartridge tube (14) and are individually pushed out of the cartridge (14).

26. Device according to claim 25, characterized thereby that
the cartridge tube (14) has a rigid jacket.

27. Device according to claim 25 or 26, characterized thereby that
the cartridge tube (14) has an elastically formed partial region (16) of the jacket in the outlet region (15) of the cartridges (9).

28. Device according to at least one of the claims 25 to 27, characterized thereby that
at least one support is arranged in the cartridge tube (14). which allows a movement of one or more cartridges towards the outlet region (15) but prevents a movement away from the outlet region (15) into the cartridge tube (14).

29. Device according to one or more of the claims 25 to 28, characterized thereby that
the cartridge tube (14) has a feed arrangement for at least one cartridge (9) operable from outside.

30. Device according to one of the claims 21 to 24, characterized thereby that
the cartridge-tube-combination is releasably arranged at the front at a pincer leg (17), whereby the tube outlet for the implant is directed towards the other pincer leg (18), which in this region is flat, plate-shaped, also convex or concave curved or is formed to be adapted to the tissue form and supports the tissue on the opposite side on pressing in of the implant.

31. Device according to one of the claims 21 to 24, characterized thereby that
the cartridge tube is disposed at the front at a pincer leg (17), whereby the tube outlet for the implant is directed towards the other pincer leg (18), which in this region is formed flat, plate-shaped or concave or convex curved or adapted to the tissue form and supports the tissue of pressing-in of the implant on the opposite side.

32. Use of the needle-type body formed as implant for stimulating acupuncture points.

33. Use of the needle-shaped body formed as implant of code carrier for identification of animals.

34. Use of the needle-shaped body formed as implant for location purposes.
